(19) United States
(12) Patent Application Publication
(10) Pub. No.: US 2014/0323832 A1
(43) Pub. Date: Oct. 30, 2014

(54) System and Method for non-invasive measurement of bilirubin in newborn and infants

(71) Applicants: Selvaraj Thangaraj, Chennai (IN); Rajiv Mohan Dhas, Chennai (IN)

(72) Inventors: Selvaraj Thangaraj, Chennai (IN); Rajiv Mohan Dhas, Chennai (IN)

(21) Appl. No.: 13/869,336

(22) Filed: Apr. 24, 2013

(51) Int. Cl. A61B 5/145; A61B 5/00; A61B 5/1455

(52) U.S. Cl. CPC A61B 5/14546; A61B 5/1455; A61B 5/7425
USPC 600/315

(57) ABSTRACT

A system for detection of bilirubin content in neonate's skin includes at least one camera configured to capture at least one image of neonate's skin and a reference image, a display system, a communication interface and a control unit configured to control functioning of other elements of the system. The control unit is further configured to detect bilirubin level by comparing image of neonate's skin and reference image. Furthermore, the control unit is configured to transfer said image of neonate's skin to at least one of communication interface and display system. The reference image is a single image acquired from any one of the dermal zones or multiple images from more than one dermal zones of neonate under observation.

The present application claims priority from Indian Application Number 1647/CHE/2012, filed on 26 Apr. 2012, the disclosure of which is hereby incorporated by reference herein.

### TECHNICAL FIELD

The embodiments herein relate to a system and a method for measuring bilirubin level, and more particularly but not exclusively to a system and a method for non-invasive measurement of bilirubin content, especially in newborns and infants.

### BACKGROUND

Bilirubin is a typical metabolic product of hemoglobin which is an oxygen carrier in blood. In the liver, the bilirubin is conjugated with glucuronic acid by the enzyme glucuronyl transferase, and then excreted through the biliary system. The enzyme glucuronyl transferase makes the bilirubin soluble in water thereby assisting the excretion process. Determination of the amount of bilirubin in body fluid, especially blood, is important for detection of hemolysis and for checking liver function. One symptom of excess bilirubin in blood is jaundice. Elevated level of bilirubin content in blood (hyperbilirubinemia) results in various other diseases.

Newborn infants, and particularly premature ones, are susceptible to the elevated level of bilirubin. Mostly, the elevated level of bilirubin in newborn infants is due to the lack of functioning glucoronyl transferase enzyme in their liver, or excessive red blood cell breakdown associated with erythroblastosis fetalis. Extreme hyperbilirubinemia places the newborn infants at risk of kernicterus, the leakage of bilirubin into the basal ganglia in the brain, and potentially causes neuronal retardation. For the aforementioned reason, it is desirable to regularly monitor the bilirubin concentrations in the body.

Jaundice or icterus is a condition caused due to the excess bilirubin in blood (hyperbilirubinemia) and often results in yellow discoloration of skin. A bilirubin level of more than 5 mg/dl manifests as clinical jaundice or icterus in neonates whereas in the adult skin 2 mg/dl of bilirubin level is high enough to cause discoloration. Further, the baby's body is divided into five dermal zones each corresponding to different values of bilirubin content that might cause jaundice. Correlation between the dermal zones and the corresponding bilirubin level for jaundice is depicted in the following table,

| | | Bilirubin level for |
|---|---|---|
| S. No | Dermal Zone | Jaundice (mg/dL) |
| 1 | Face | 4-8 |
| 2 | Upper Trunk | 5-12 |
| 3 | Lower Trunk | 8-16 |
| 4 | Arms of lower legs | 11-18 |
| 5 | Palms and Soles | Greater than 15 |

Clinical Jaundice first becomes visible in the face. Further, as the bilirubin content in the baby's blood increases proportionately the yellowish discoloration begins from head and extends towards the feet. The bilirubin level is expected to be greater than 15 mg/dl if the soles are found icteric.

The conventional method for detecting jaundice or icterus in infants is by blanching the skin with finger pressure over a bony part. However, the conventional method results in revealing the underlying skin and subcutaneous tissue. Further, the accuracy of results obtained from the conventional method depends upon observer's skill, that varies from person to person and the environmental conditions such as brightness at the time of detecting the bilirubin content and so on. Furthermore, while detecting icterus for multiple infants, the observer may not be able to keep a note on the bilirubin content of each and every infant and hence a future analysis may not be possible. Further, patient identification and logging of the data against the patient's records may not be possible in the conventional method.

The other methods for detecting the bilirubin concentration in the blood are heel prick method and an optical system. In heel prick method, blood samples are taken from neonate for detecting the bilirubin concentration. However, this method is an invasive technique for measurement of bilirubin concentration in the skin. The latter is a non-invasive technique which uses the optical method for detecting the bilirubin concentration in the blood. The cost of such (existing) non-invasive devices is high. Therefore, there is a need for developing a non-invasive system for measuring the bilirubin concentration at an affordable price. Further, there is a need to provide a system for detecting the bilirubin content, which is not affected by environmental conditions and is capable of obviating the above mentioned drawbacks in the conventional methods.

### SUMMARY

The principal object of this embodiment is to provide a system for non-invasive detection of bilirubin content.

Another object of the embodiment is to provide a controlled environment for the bilirubin content detection.

A further object of the embodiment is to transmit the image of the infant to remote physicians for diagnosis.

A further object of the embodiment is to provide a system that could enable patient identification and logging of the data against the patient's medical records.

Yet another object of the embodiment is to provide a method for non-invasive detection of bilirubin content.

These and other objects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF FIGURES

This embodiment is illustrated in the accompanying drawings, throughout which reference letters indicate corresponding parts in the various figures. The embodiments herein will be better understood from the following description with reference to the drawings, in which:

FIG. 1 shows a block diagram of a system for non-invasive detection of bilirubin content according to an embodiment of the present embodiment; and

FIG. 2 is a flow chart depicting a method for non-invasive detection of bilirubin content according to an embodiment of the present embodiment.

### DETAILED DESCRIPTION OF EMBODIMENT

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

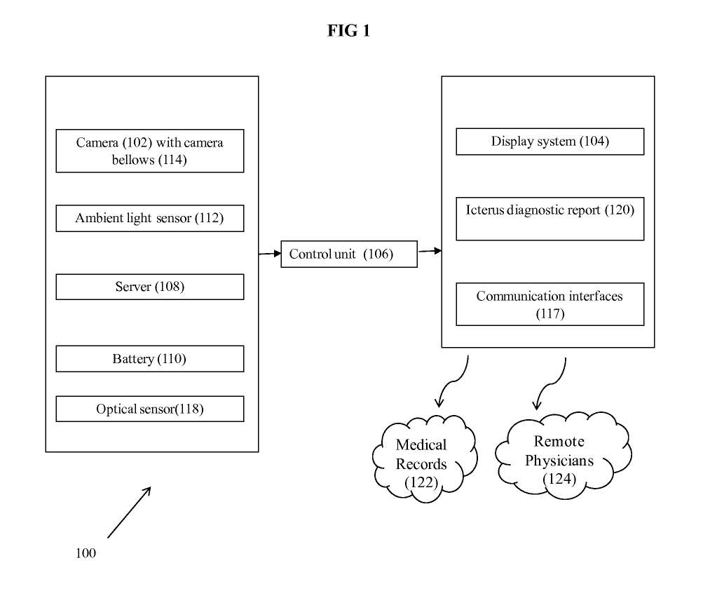

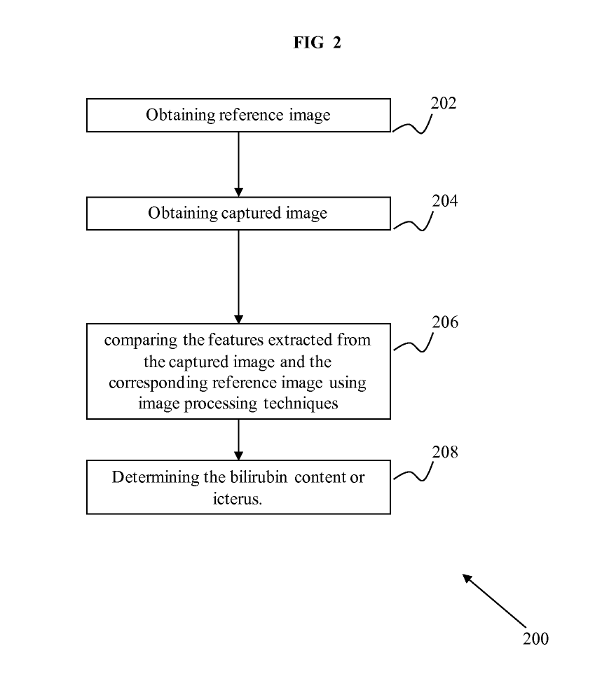

The embodiments herein provide a system for non-invasive detection of bilirubin level according to an embodiment of the present embodiment. Referring now to the drawings, and more particularly to FIGS. 1 and 2, where similar reference characters denote corresponding features consistently throughout the figures, there are shown embodiments.

FIG. 1 shows a block diagram of a system for non-invasive detection of bilirubin level according to an embodiment of the present embodiment. The system 100 includes a camera 102, a display system 104, a control unit 106, a server 108, a battery 110, an ambient light sensor 112, an Optical sensor 118 and a plurality of camera bellows 114. The camera 102 is configured to capture image of a neonate skin, which is subjected to diagnostic analysis. In one embodiment, the image capturing is performed on a suitable area on the face, preferably nose portion. Further, camera 102 is configured to capture a reference image. The reference image is a single image acquired from any one of the dermal zones or multiple images from more than one dermal zone. The dermal zone constitutes five different zones in a neonate body, each corresponding to different values of bilirubin. Further, the camera 102 includes a light emitting diode or other suitable light sources which is configured to provide necessary light to capture images. Further, camera bellows 114 are configured to provide a controlled environment for capturing the image. In one embodiment, the camera bellow 114 is selected from a group that includes but are not limited to a cloth or leather bellow that keeps the light out. However, it is also within the scope of the embodiment that the camera bellow 114 may be selected from any other material or adapters without otherwise deterring intended function of camera bellow 114 as can be deduced from this description. Further, camera bellow 114 acts as a shield to camera 102, thereby preventing ambient light from entering into the field of image acquisition. The prevention of ambient light from entering into the field of image acquisition provides a controlled environment for acquiring the images. In one embodiment, the ambient light sensor 112 is provided to sense the presence of ambient light within the field of image acquisition. Further, the control unit 106 is configured to alert the user of system 100, if ambient light is present within the field of image acquisition.

Further, the control unit 106 is configured to regulate functioning of all the other elements of system 100. The control unit 106 includes a programmable controller, a memory and input/output peripherals. The input peripheral of the control unit 106 is provided in communication with the camera 102, ambient light sensor 112, optical sensor 118 and server 108. The output peripheral is provided in communication with the display system 104 and communication interfaces (117). The memory unit provided in the control unit 106 is configured to receive reference image from the camera 102 and store the reference image. In another embodiment, the server 108 is provided in communication with camera 102 and configured to store the reference image obtained from camera 102. The programmable controller of control unit 106 is configured to receive image of neonate skin that has to be deducted for icteric (captured image) from camera 102. The programmable controller is programmed to compare captured image and the reference image using features extracted from the image using image comparison techniques in order to determine the bilirubin level of the neonate. Further, the bilirubin level is displayed in the display system 104 provided in the output peripheral of the control unit 106. In one embodiment, the pixel value of the captured image and the reference image are compared. However, it is also within the scope of embodiment that the image comparison techniques will include the comparison of any other features of the reference image and the captured image without otherwise deterring intended function of the system as can be deduced from this description. Further, the control unit 106 is configured to transfer the image of neonate to the remote physicians for diagnosis 124 via communication interfaces 117. In an embodiment, the control unit 106 is configured to transfer the image of neonate to a display system 104. Further, the display system 104 is adapted to display the image of neonate. In one embodiment, the display system 104 is provided in communication with the camera 102. In another embodiment, an optical sensor 118 is provided in communication with the control unit 106. The optical sensor 118 identifies the neonate under diagnostic analysis using optical reader capabilities. Further, the control unit 106 is configured to record the diagnostic result of a neonate obtained from the image comparison technique into corresponding neonate's icterus diagnostic report 120 and also transfer the data (level of bilirubin content) to the neonate's medical records 122 via communication interface (wired interfaces like USB, LAN/Ethernet, and wireless interfaces like Bluetooth, WiFi, 3 G/GPRS) 117 using the patient identification number. Further, a battery 110 is configured to provide power supply to all the elements of the system 100.

It should be noted that the aforementioned configuration of system 100 is provided for the ease of understanding of the embodiments of the embodiment. However, certain embodiments may have a different configuration of the components of the system 100 and certain other embodiments may exclude certain components of the system 100. For example, plurality of camera 102 may be provided in the system, for obtaining the reference image and the captured image separately. Further, the control unit 106 may include any other hardware device, combination of hardware devices, software devices or combination of hardware or software devices that could achieve one or more processes discussed below. Further, the control unit 106 may include certain other elements such as an image control unit that is configured to control the functionality of camera 102, LED/other suitable light source's control unit that is configured to control the functionality of LED/other suitable light source provided with the camera 102 and battery management unit that is configured to manage the battery 110. Further, the server 108 can be removed from the system 100, so that the system 100 may be used as a standalone device with the reference image being stored at the inbuilt memory unit. Further, the system 100 could be operated with a limited bandwidth of patients in rural settings or primary healthcare units. Therefore, such embodiments and any modification by addition or exclusion of certain components of the system 100 without otherwise deterring the intended function of the system 100 as is apparent from this description and drawings are also within the scope of this embodiment.

The method (200) for non-invasive measurement of bilirubin in newborn and infants is explained herein below. FIG. 2 is a flow chart depicting a method for non-invasive detection of bilirubin content according to an embodiment of the present embodiment. The method includes acquiring a reference image (step 202). The reference image is a single image acquired from any one of the dermal zones or multiple images from more than one dermal zones of neonate who is under observation. The dermal zone is present in five different zones in a neonate's body, each corresponding to different values of bilirubin that might cause jaundice. The reference image is acquired from camera 102. The camera bellow 114 provides a controlled environment for reference image capturing. In one embodiment, the camera bellow 114 is selected from a group that includes but are not limited to a cloth or leather bellow that keeps the light out. However, it is also within the scope of the embodiment that the camera bellow 114 may be selected from any other material or adapters without otherwise deterring intended function of camera bellow 114 as can be deduced from this description. Further, camera bellow 114 acts as a shield to camera 102, thereby preventing ambient light from entering into the field of image acquisition. The prevention of ambient light from entering into the field of image acquisition provides a controlled environment for acquiring image. Further, the reference image obtained is stored in a memory unit or server through network capability. Further, for obtaining reference image, the user blanches the skin of the neonate under observation, and places the camera 102 over the blanched area to obtain the image. In an embodiment, Light Emitting Diode or other suitable light sources may be configured to provide the necessary light for obtaining the images. The image acquisition is done in a controlled environment using the camera bellows or similar adapters which can limit the ambient light from entering the field. The acquired image is compared with the gosset icterometer value before storing it as reference image. The acquired image is taken as reference image and stored either in server 108 via communication interface 117 or in the memory unit of control unit 106.

Further, the system 100 obtains the image of a neonate skin (captured image), under diagnostic analysis using camera 102 (step 204). The captured image is obtained by blanching the skin of a neonate under diagnostic analysis and placing camera 102 over the blanched area to obtain the image. In an embodiment, the captured image is obtained in a controlled environment using camera bellows or similar adapters which can limit ambient light from entering into the field of image acquisition. In an embodiment, Light emitting diode or other suitable light sources may be configured to provide the necessary light for obtaining the images. Further, the control unit 106 is programmed to determine the presence of icteric by comparing the features extracted from the captured image and the corresponding reference image using image processing techniques (step 206). In one embodiment, the pixel value of the captured image and the reference image is compared. However, it is also within the scope of embodiment that the image comparison techniques will include the comparison of any other features of the reference image and the captured image without otherwise deterring intended function of the system as can be deduced from this description. Further, if the difference between the reference image and the captured image exceeds a threshold value, then the neonate is considered to be icteric (step 208). In one embodiment, the pixel value of the captured image is checked with gosset icterometer pixel values for cross reference. In another embodiment, the captured image is obtained from suitable area on face, preferably nose. Further, the image features are extracted from the captured image using image processing techniques. The extracted features are compared with the reference image and gosset icterometer features. Further, the program enabled in control unit 106 calculates specific value or bilirubin level based on the on the image features. Furthermore, the control unit 106 enables the display system 104 to display image of the neonate obtained from camera 102. Further, the optical sensor 118 identifies the neonate under diagnostic analysis using optical reader capabilities. Further, the control unit 106 records the diagnostic result of a neonate obtained from the image comparison technique into the corresponding neonate's icterus diagnostic report 120 and also transfers it to the neonate's medical records 122 via communication interface 117 using the patient identification number. As is evident from the above description, with the system 100 and the method disclosed herein, the objectives as was set forth initially will be achieved.

It should be noted that the aforementioned steps for performing the method for non-invasive measurement of bilirubin in newborn and infants are provided for the ease of understanding of the embodiments of the embodiment. However, various steps provided in the above method may be performed in the order presented, in a different order, or simultaneously. Further, in some embodiments, one or more steps listed in the above method may be omitted. Therefore, such embodiments and any modification that is apparent from this description and drawings are also within the scope of this embodiment.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

We claim:

1. A system for detection of bilirubin content in neonate's skin, said system comprising:
at least one camera configured to capture at least one image of neonate's skin and a reference image;
a display system;
a communication interface; and
a control unit configured to control functioning of at least one of said camera, said display system and said communication interface,
wherein
said reference image is a single image acquired from any one of the dermal zones or multiple images from more than one dermal zones of neonate under observation;
said control unit is configured to detect bilirubin level by comparing said image of neonate's skin and said reference image; and
said control unit is configured to transfer said image of neonate's skin to at least one of communication interface and display system.

2. The system as claimed in claim 1, wherein said camera includes a light source that is configured to provide necessary light to capture at least one of image of neonate's skin and reference image.

3. The system as claimed in claim 1 further includes at least one camera bellow configured to prevent entry of ambient light into a camera's field of image acquisition.

4. The system as claimed in claim 1, wherein said system further comprising:
an optical sensor having optical reader capabilities to identify neonate under observation; and
at least one of neonate's Icterus diagnostic report and medical records,
wherein
said control unit is configured to record said detected bilirubin level into corresponding said neonate's Icterus diagnostic report and said medical records via communication interface.

5. The system as claimed in claim 1 further includes an ambient light sensor configured to sense the presence of ambient light within said camera's field of image acquisition, wherein said control unit is configured to alert user of system regarding said ambient light present within camera's field of image acquisition.

6. A method for non-invasive measurement of bilirubin level, wherein said method comprising:
obtaining at least one reference image and image of neonate's skin in a controlled environment;
storing said reference image; and
extracting the features of obtained reference image and image of neonate's skin,
wherein
bilirubin level is measured by comparing said extracted features of image of neonate's skin and reference image; and
said reference image is a single image acquired from any one of the dermal zones or multiple images from more than one dermal zones of neonate under observation.

7. The method as claimed in claim 6 further includes comparing the reference image with a gosset icterometer value before storing said reference image.

8. The method as claimed in claim 6, wherein said method further comprises of:
Identifying the said neonate using optical reader capabilities; and
recording said measured bilirubin level into corresponding at least one of neonate's icteric diagnostic report and medical records.

9. The method as claimed in claim 6, wherein said controlled environment is provided by preventing entry of ambient light while obtaining at least one of reference image and image of neonate's skin.

10. The method as claimed in claim 6 further includes transferring said obtained image of neonate's skin via at least one communication interface; and displaying said obtained image of neonate's skin on an electronic display device, for in care-premises or remote physicians for diagnosis.
